(19) United States
(12) Patent Application Publication
(10) Pub. No.: US 2024/0159419 A1
(43) Pub. Date: May 16, 2024

(54) DETERMINATION SYSTEM, SENSOR, DETERMINATION METHOD, AND RECORDING MEDIUM

(71) Applicant: PANASONIC INTELLECTUAL PROPERTY MANAGEMENT CO., LTD., Osaka (JP)

(72) Inventors: Etsuko MIZUNO, Osaka (JP); Yasuo KOHASHI, Osaka (JP); Taiji SASAKI, Osaka (JP)

(21) Appl. No.: 17/780,708

(22) PCT Filed: Jun. 17, 2021
(86) PCT No.: PCT/JP2021/023110

§ 371 Date: Sep. 1, 2022

(30) Foreign Application Priority Data
Feb. 24, 2021 (JP) 2021-027994

(51) Int. Cl. F24F 11/67; F24F 11/52; F24F 120/14

(52) U.S. Cl. CPC F24F 11/67; F24F 11/52; F24F 2120/14

(57) ABSTRACT

A determination system includes an obtainer, a determiner, and an outputter. The obtainer obtains a result of detection by a first sensor which detects a body movement of a body part of a user exposed from a covering, and a result of detection by a second sensor which detects a body movement of the user. The determiner determines a sleep state of the user, based on the result of detection by the first sensor and the result of detection by the second sensor, which are obtained by the obtainer, the sleep state including a state of the covering. The outputter outputs information based on a result of determination by the determiner.

### CROSS-REFERENCE OF RELATED APPLICATIONS

This application is the U.S. National Phase under 35 U.S.C. § 371 of International Patent Application No. PCT/JP2021/023110, filed on Jun. 17, 2021, which in turn claims the benefit of Japanese Patent Application No. 2021-027994, filed on Feb. 24, 2021, the entire disclosures of which applications are incorporated by reference herein.

### TECHNICAL FIELD

The present disclosure relates to a determination system which determines the state of a user when sleeping, a sensor, a determination method, and a recording medium.

### BACKGROUND ART

Patent Literature (PTL) 1 discloses an air-conditioning system. This air-conditioning system includes a head movement amount measurer which measures the movement amount of the head of a sleeper, a body movement amount measurer which measures the movement amount of the body of the sleeper, and a controller. The controller calculates a difference between the movement amounts of these parts measured by the corresponding measurers. When the difference is less than or equal to a threshold, the controller transmits an abnormality signal to an informer to inform another person or the sleeper that the sleeper is out of the bedding.

### CITATION LIST

### Patent Literature

[PTL 1] Japanese Unexamined Patent Application Publication No. 2007-120815

### SUMMARY OF INVENTION

### Technical Problem

The present disclosure provides a determination system and the like which facilitates provision of an environment suitable for a user when sleeping.

### Solution to Problem

The determination system according to one aspect of the present disclosure includes an obtainer, a determiner, and an outputter. The obtainer obtains a result of detection by a first sensor which detects a body movement of a body part of a user exposed from a covering, and a result of detection by a second sensor which detects a body movement of the user. The determiner determines a sleep state of the user, based on the result of detection by the first sensor and the result of detection by the second sensor, which are obtained by the obtainer, the sleep state including a state of the covering. The outputter outputs information based on a result of determination by the determiner.

The sensor according to one aspect of the present disclosure has a communication function to communicate with the determination system, and a detection function to detect the body movement of the user, and the sensor transmits a result of detection using the detection function to the determination system using the communication function.

The determination method according to one aspect of the present disclosure includes obtaining, determining, and outputting. The obtaining includes obtaining a result of detection by a first sensor which detects a body movement of a body part of a user exposed from a covering, and a result of detection by a second sensor which detects a body movement of the user. The determining includes determining a sleep state of the user, based on the result of detection by the first sensor and the result of detection by the second sensor, which are obtained in the obtaining, the sleep state including a state of the covering. The outputting includes outputting a result of determination in the determining.

The recording medium according to one aspect of the present disclosure is a non-transitory computer-readable recording medium for use in a computer, the recording medium having a computer program recorded thereon for causing one or more processors to execute the determination method.

### Advantageous Effects of Invention

The determination system according to the present disclosure and the like advantageously facilitate provision of an environment suitable for a user when sleeping.

### BRIEF DESCRIPTION OF DRAWINGS

FIG. 1 is a block diagram illustrating an overall configuration including the determination system according to an embodiment.

FIG. 2 is a schematic diagram illustrating one example of a space in which the determination system according to the embodiment is used.

FIG. 3 is a table listing the information indicating a correlation with the Clo value.

FIG. 4 is a graph showing the results of a first experiment.

FIG. 5 is a graph showing the results of a second experiment.

FIG. 6 is a graph showing the results of a third experiment.

FIG. 7 is a graph showing one example of the correlation between the Clo value and the comfort temperature.

FIG. 8 is a flowchart illustrating an example of an operation of the determination system according to the embodiment.

FIG. 9 is a block diagram illustrating an overall configuration including the determination system according to Modification 1 of the embodiment.

### DESCRIPTION OF EMBODIMENT

### (Underlying Knowledge Forming Basis of the Present Disclosure)

First, the viewpoint of the inventor will be described below.

When an air-conditioning apparatus is used during sleeping, irrespective of the seasons, the setting (such as the room temperature or the air volume) of the air-conditioning apparatus such that a user feels comfortable can be varied depending on the cloth that the user wears and/or the bedding that the user uses.

However, it is difficult for the user to know the optimal setting of the cloth to wear and/or the bedding to use. For this reason, the user only performs the basic setting, for example, the user sets the room temperature at 27° C. in summer and at 20° C. in winter. In this case, the air-conditioning apparatus does not successfully provide an environment suitable for the user, thus often leading to unsatisfaction of the user.

In consideration of such circumstances, the inventor has devised the present disclosure.

Hereinafter, an embodiment will be described in detail with reference to the drawings. However, detailed description beyond necessity will be omitted in some cases. For example, detailed descriptions of already well-known things and overlapping descriptions of substantially identical configurations will be omitted in some cases. This aims at avoiding unnecessary redundancy in the description below and facilitating understanding by persons skilled in the art.

The inventor provides the accompanying drawings and the description below for sufficient understanding of the present disclosure by persons skilled in the art, and does not intend to limit the subject matter specified in Claims by these.

### EMBODIMENT

### [1-1. Overall Configuration]

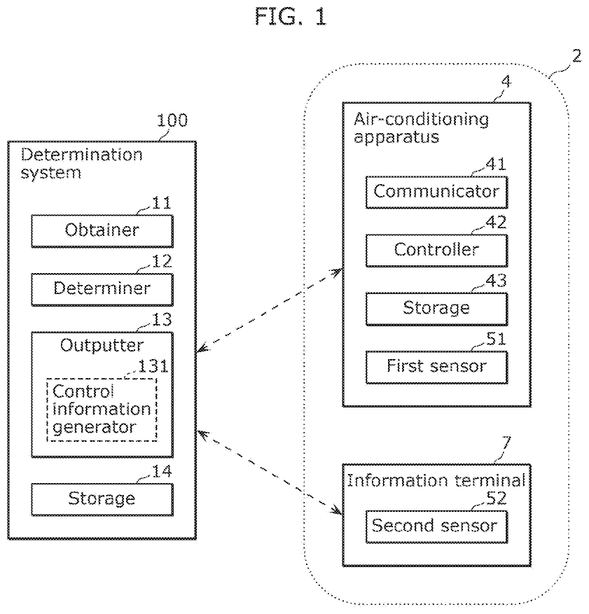

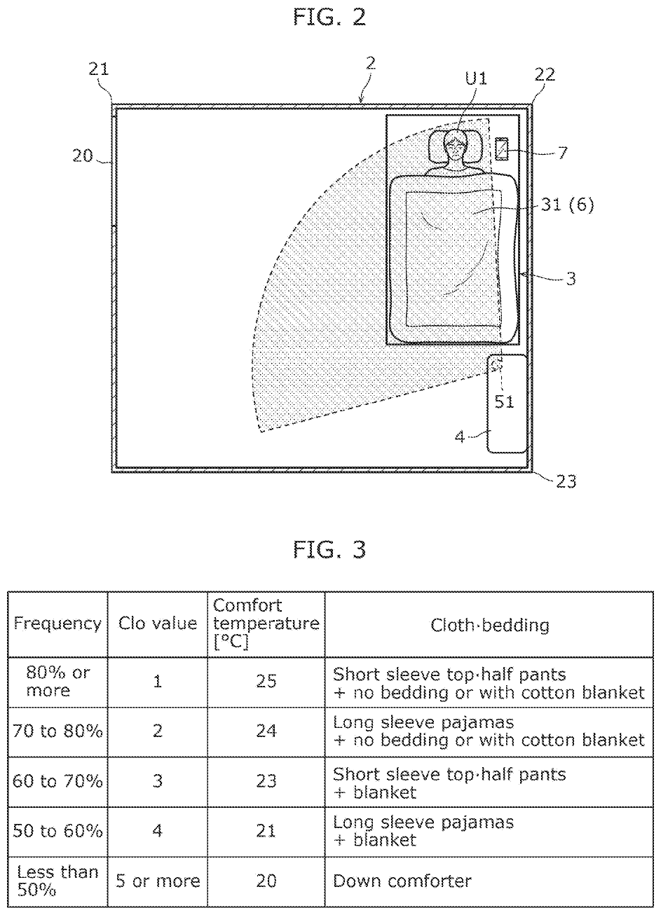

First, the overall configuration including determination system 100 according to an embodiment will be described with reference to FIGS. 1 and 2. FIG. 1 is a block diagram illustrating the overall configuration including determination system 100 according to the embodiment. FIG. 2 is a schematic diagram illustrating one example of space 2 in which determination system 100 according to the embodiment is used.

In the example illustrated in FIG. 2, space 2 corresponds to room 2 used by user U1 at least when sleeping. Room 2 has a rectangular shape in a planar view, and includes entrance 20 to room 2 in first corner 21 (the upper left corner in FIG. 2) among the four corners. Bed 3 is disposed in second corner 22 (the upper right corner in FIG. 2) among the four corners of room 2. Furthermore, air-conditioning apparatus 4 is disposed in third corner 23 (the lower right corner in FIG. 2) among the four corners of room 2.

Bed 3 is a place where user U1 sleeps. In the embodiment, sleeping place 3 includes a bed disposed on the floor in room 2. Sleeping place 3 may be a place on which user U1 can lie down to sleep, and may be the floor itself in room 2, for example.

In sleeping place 3, bedding 31 used by user U1 can be disposed. Bedding 31 may include a first bedding member and a second bedding member. The first bedding member is the bedding which is disposed between user U1 and sleeping place 3 during sleeping and on which user U1 lies down. Examples of the first bedding member may include a pillow, a sheet, a mattress, and a futon mattress. The second bedding member is the bedding which is disposed over user U1 during sleeping to cover part of user U1 (for example, the body other than the head). Examples of the second bedding member may include a cotton blanket, a blanket, and a comforter.

When user U1 uses bedding 31, bedding 31 may include at least one of the first bedding member or the second bedding member. For example, some users U1 may use only a sheet (first bedding member) in sleeping place 3 but not the second bedding member such as a comforter. Moreover, bedding 31 is not always disposed in sleeping place 3. For example, some users U1 may sleep in sleeping place 3 without using bedding 31.

Air-conditioning apparatus 4 is an air-conditioner installed on a wall of room 2, for example, and controls the temperature (room temperature) of room 2 to the set temperature by blowing the air having a controlled temperature to room 2. In the embodiment, air-conditioning apparatus 4 can operate both in a cooling mode and a heating mode. To be noted, air-conditioning apparatus 4 may be an apparatus which can operate only in a cooling mode, or may be an apparatus which can operate only in a heating mode.

Air-conditioning apparatus 4 includes communicator 41, controller 42, and storage 43. In the embodiment, air-conditioning apparatus 4 further includes first sensor 51. First sensor 51 will be described in [1-2. Determination system] described later

Communicator 41 receives a first control signal by communicating with a remote controller for air-conditioning apparatus 4, the first control signal including a command according to an operation input accepted by the remote controller. For example, communicator 41 receives the first control signal by communicating with the remote controller using infrared light as a medium. The communication between communicator 41 and the remote controller is not limited to infrared light communication, and may be wireless communication using radio waves as a medium, for example.

Moreover, communicator 41 receives a second control signal transmitted by outputter 13, by communicating with outputter 13 (described later) of determination system 100. Communicator 41 receives the second control signal by communicating with outputter 13 via an external network such as the Internet. The communication between communicator 41 and outputter 13 may be wired communication as well as wireless communication. Any standard can be used in the communication between communicator 41 and outputter 13.

Controller 42 is a microcomputer, for example, and implements a variety of functions by a processor which executes a computer program stored in storage 43. In the embodiment, based on the first control signal received by communicator 41, controller 42 controls the temperature (room temperature) of room 2 to the set temperature designated by the first control signal. Based on the second control signal received by communicator 41, controller 42 controls the temperature of room 2 to the set temperature designated by the second control signal. When communicator 41 receives the second control signal while controller 42 is performing the control based on the first control signal, controller 42 preferentially performs the control based on the second control signal.

Storage 43 is a memory device which stores information (such as a computer program) needed for a variety of controls performed by controller 42. Although storage 43 is implemented by a semiconductor memory, for example, a known electronic device for storing Information can be used without limitation. Storage 43 stores the set temperature designated by the first control signal or the second control signal, for example.

### [1-2. Determination System]

Next, details of determination system 100 will be described. As illustrated in FIG. 1, determination system 100 includes obtainer 11, determiner 12, outputter 13, and storage 14. In the embodiment, it is sufficient that determination system 100 includes at least obtainer 11, determiner 12, and outputter 13, and may not include storage 14.

In the embodiment, determination system 100 is configured with a server located in a place remote from the facility including room 2 in which user U1 sleeps. Alternatively, determination system 100 may be disposed in the facility. In the embodiment, an example will be described, in which one user U1 is focused on and determination system 100 determines the sleep state of user U1.

Obtainer 11 obtains the result of detection by first sensor 51 by communicating with first sensor 51. For example, obtainer 11 obtains the result of detection by first sensor 51 by communicating with first sensor 51 according to the wireless communication standard such as Wi-Fi (registered trademark) or Bluetooth (registered trademark) Low Energy (BLE). The communication between obtainer 11 and first sensor 51 may be wired communication as well as wireless communication. Any standard can be used in the communication between obtainer 11 and first sensor 51.

First sensor 51 detects the body movement of a body part of user U1 exposed from covering 6. Here, covering 6 is disposed over user U1. As one example, covering 6 may include a cloth that user U1 wears or bedding 31 (second bedding member) that user U1 uses. In the embodiment, first sensor 51 is a pyroelectric infrared sensor, and detects infrared light radiated from user U1. First sensor 51 detects a change in detection level as a body movement of user U1. This is because the detection level of first sensor 51 is varied in response to a movement of part (such as the head and/or arms and legs) of user U1 exposed from covering 6.

In the embodiment, first sensor 51 is attached to air-conditioning apparatus 4. In the embodiment, the detection range of first sensor 51 partially or completely includes sleeping place 3 (see the dot hatched region in FIG. 2). In other words, it is sufficient that first sensor 51 is installed such that part of user U1 exposed from covering 6 is included in the detection range thereof.

Obtainer 11 obtains the result of detection by second sensor 52 by communicating with second sensor 52. For example, obtainer 11 obtains the result of detection by second sensor 52 by communicating with second sensor 52 according to the wireless communication standard such as Wi-Fi (registered trademark) or Bluetooth (registered trademark) Low Energy (BLE). The communication between obtainer 11 and second sensor 52 may be wired communication as well as wireless communication. Any standard can be used in the communication between obtainer 11 and second sensor 52.

Second sensor 52 detects the body movement of user U1. Here, unlike first sensor 51, second sensor 52 detects the body movement of user U1 irrespective of the presence/absence of covering 6. In the embodiment, second sensor 52 is a vibration sensor, an acceleration sensor, or a piezoelectric sensor, and indirectly detects the body movement of user U1 by detecting vibration of sleeping place 3 or the first bedding member, on which second sensor 52 is disposed. In the embodiment, second sensor 52 is an acceleration sensor as one example. A body movement of user U1 vibrates sleeping place 3 or the first bedding member, on which user U1 sleeps. Second sensor 52, when attached to the cloth of user U1 or the like, may directly detect the body movement of user U1.

In the embodiment, second sensor 52 is built in information terminal 7 possessed by user U1. In other words, in the embodiment, it is presumed that user U1 sleeps while information terminal 7 is placed on sleeping place 3 or the first bedding member. Information terminal 7 may include a smartphone or a tablet terminal, for example.

Determiner 12 determines the sleep state of user U1 including covering 6, based on the result of detection by first sensor 51 and the result of detection by second sensor 52, which are obtained by obtainer 11. In other words, determiner 12 determines the type of cloth which user U1 wears and/or the type of bedding 31 which user U1 uses. For example, determiner 12 determines whether user U1 wears a short sleeve cloth or a long sleeve cloth. For example, determiner 12 determines whether bedding 31 (second bedding member) used by user U1 is lightweight or heavyweight.

The present inventor has found that the Clo value is correlated with a combination of the body movement of a body part of user U1 exposed from covering 6 during sleeping and the body movement of user U1 during sleeping irrespective of covering 6. Thus, in the embodiment, determiner 12 determines the sleep state of user U1 by calculating the Clo value based on the result of detection by first sensor 51 and the result of detection by second sensor 52 obtained by obtainer 11.

Here, the Clo value is an index indicating thermal insulating properties and heat-retaining properties of clothing worn by a user, and is advocated by American Society of Heating, Refrigerating and Air-Conditioning Engineers (ASHRAE). A Clo value of “1” is defined as heat-retaining properties of a cloth to wear such that an adult male feels comfortable and can maintain the average skin temperature at 33° C. when he calmly sits in a chair in a room having an air temperature of 21° C., a relative humidity of 50% or less, and an air flow rate of 0.1 m/s or less. In the embodiment, the Clo value is used as an index indicating the type of cloth which user U1 wears and/or the type of bedding 31 which user U1 uses. The Clo value used in the embodiment is a uniquely converted index including the bedding amount, rather than a standard index for a cloth.

FIG. 3 shows one example of the correlation between the Clo value and covering 6. FIG. 3 is a table listing the Clo value and its correlated information. As shown in FIG. 3, when the Clo value is “1”, it corresponds to a combination of a short sleeve top and half pants without bedding 31 (second bedding member) or with relatively lightweight bedding having low heat-retaining properties, such as a cotton blanket, or clothes and/or bedding 31 having heat-retaining properties similar to those of these. When the Clo value is “2”, it corresponds to a combination of long sleeve pajamas without bedding 31 or with relatively lightweight bedding having low heat-retaining properties, such as a cotton blanket, or clothes and/or bedding 31 having heat-retaining properties similar to those of these. When the Clo value is “3”, it corresponds to a combination of a short sleeve top and half pants with bedding 31 which is relatively lightweight and has high heat-retaining properties, such as a blanket, or clothes and/or bedding 31 having heat-retaining properties similar to those of these. Shen the Clo value is “4”, it corresponds to a combination of long sleeve pajamas with bedding 31 which is relatively lightweight and has high heat-retaining properties, such as a blanket, or clothes and/or bedding 31 having heat-retaining properties similar to those of these. When the Clo value is “5 or more”, it corresponds to a combination of any cloth with bedding 31 which is relatively heavyweight and has high heat-retaining properties, such as a down comforter, or bedding 31 having similar to those of this bedding 31.

In the embodiment, determiner 12 determines the sleep state of user U1, based on the result of comparison between the frequency of detection of the body movement of user U1 by second sensor 52 and that of detection of the body movement of user U1 by first sensor 51. In particular, here, determiner 12 determines the sleep state of user U1, based on the frequency of detection of the body movement of user U1 by first sensor 51 in a period in which second sensor 52 detects the body movement of user U1. In other words, the frequency is the proportion of the number of body movements of user U1 simultaneously detected by first sensor 51 and second sensor 52 to the number of body movements of user U1 detected by second sensor 52. In the embodiment, the frequency is expressed in percentage. In short, determiner 12 determines the sleep state of user U1 by calculating the frequency based on the result of detection by first sensor 51 and the result of detection by second sensor 52, and calculating the Clo value corresponding to the calculated frequency with reference to the data shown in FIG. 3.

FIG. 3 shows one example of the correlation between the frequency and the Clo value. As shown in FIG. 3, determiner 12 calculates the Clo value as “1” when the frequency is 80% or more, as “2” when the frequency is 70% or more and less than 80%, and as “3” when the frequency is 60% or more and less than 70%. Determiner 12 calculates the Clo value as “4” when the frequency is 50% or more and less than 60%, and the Clo value as “5 or more” when the frequency is less than 50%.

The correlation between the frequency and the Clo value is one example, and is not limited to this. For example, the range of the frequency corresponding to the Clo value may be different from that shown in FIG. 3. For example, determiner 12 may calculate the Clo value while further dividing the frequency into smaller ranges. The range of the frequency corresponding to the Clo value may be slightly varied according to the seasons.

Hereinafter, the results of experiments by the present inventor about the correlation between the sleep state of user U1 and the result of detection by first sensor 51 and the result of detection by second sensor 52 will be described. The experiments were performed in space (room) 2 illustrated in FIG. 2.

In a first experiment, user U1 slept while wearing a short sleeve top and half pants. User U1 used a cool touch sheet as the first bedding member but not the second bedding member. Thus, in the first experiment, the arms and legs of user U1 were always exposed from the cloth and the bedding during sleeping.

In a second experiment, user U1 used a heavyweight down comforter as the second bedding member. In the second experiment, the cloth of user U1 is not particularly specified. In the second experiment, the arms and legs of user U1 were not exposed from the cloth and the bedding all the time during sleeping.

In a third experiment, user U1 slept while wearing long sleeve pajamas. In addition, user U1 used a brushed sheet as the first bedding member and a blanket as second bedding member 31. In the third experiment, the arms and legs of user U1 were not exposed from the cloth and the bedding all the time during sleeping.

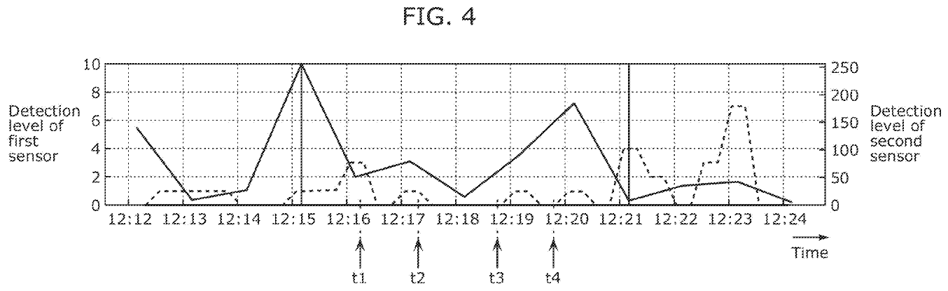

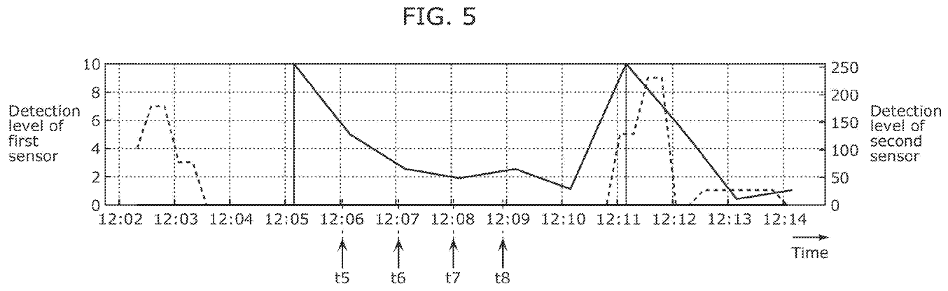

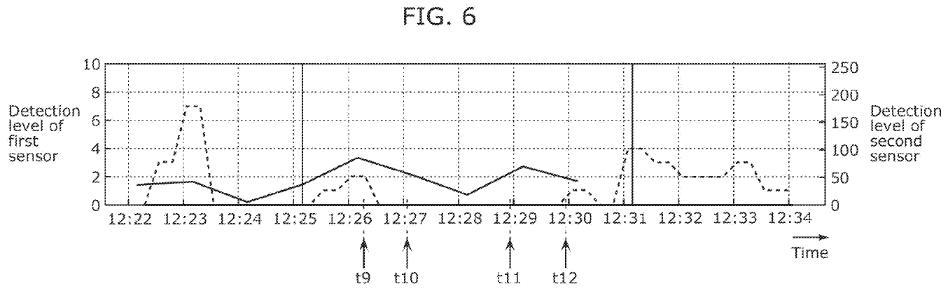

FIGS. 4 to 6 show the results of the first to third experiments. FIG. 4 is a graph showing the results of the first experiment. FIG. 5 is a graph showing the results of the second experiment. FIG. 6 is a graph showing the results of the third experiment. In each of FIGS. 4 to 6, the left ordinate represents the detection level of first sensor 51, the right ordinate represents the detection level of second sensor 52, and the abscissa represents the time. In FIG. 4, times t1, t2, t3, and t4 represent a time at which user U1 intentionally rolled over in predetermined period T1. Times t5, t6, t7, and t8 in FIG. 5 and times t9, t10, t11, and t12 in FIG. 6 are also defined as above. Furthermore, in each of FIGS. 4 to 6, the dashed line graph represents transition of the detection level of first sensor 51, and the solid line graph represents transition of the detection level of second sensor 52.

In FIGS. 4 to 6, when the detection level of first sensor 51 is more than a predetermined value (here, zero), this indicates that first sensor 51 detects the body movement of user U1. In FIGS. 4 to 6, when the slope of the detection level of second sensor 52 changes, this indicates that second sensor 52 detects the body movement of user U1.

In the first experiment, as shown in FIG. 4, first sensor 51 and second sensor 52 detect the body movement of user U1 at all of times t1, t2, t3, and t4 in predetermined period T1. In other words, in the first experiment, the frequency is 100%. It is considered that the experimental results are attributed to exposure of the arms and legs of user U1, which facilitates the detection of the body movement of user U1 not only by second sensor 52 but also by first sensor 51.

In the second experiment, as shown in FIG. 5, second sensor 52 detects the body movement of user U1 at all of times t5, t6, t7, and t8, while first sensor 51 does not detect the body movement of user U1. In other words, in the second experiment, the frequency is 0%. It is considered that the experimental results are attributed to covering of user U1 with a heavyweight down comforter, which results in difficulties in detecting the body movement of user U1 by first sensor 51.

In the third experiment, as shown in FIG. 6, second sensor 52 detects the body movement of user U1 at all of times t9, t10, t11, and t12, while first sensor 51 detects the body movement of user U1 only at times t9 and t12. In other words, in the third experiment, the frequency is 50%. It is considered that the experimental results are attributed to covering of user U1 as in the second experiment with a lightweight blanket rather than the down comforter, which facilitates the detection of the body movement of user U1 by first sensor 51 compared to the second experiment.

As shown in the first to third experiments, a correlation is found among the result of detection by first sensor 51, the result of detection by second sensor 52, and the state of user U1 during sleeping (here, the type of covering 6).

In the embodiment, determiner 12 determines the sleep state of user U1 within a predetermined time (e.g., 1 hour) from the time when user U1 sleeps. Here, the timing at which user U1 starts sleeping may be a time when information terminal 7 accepts an input indicating the start of sleep by an operation of information terminal 7 by user U1, for example. In this case, determiner 12 can grasp the time through communication with information terminal 7.

Alternatively, for example, the timing at which user U1 starts sleeping may be a time when an illuminance sensor included in air-conditioning apparatus 4 detects illuminance less than or equal to a threshold (in other words, the time when a light fixture in room 2 is turned off) while first sensor 51 is detecting the presence of user U1. In this case, determiner 12 can grasp the time through communication with first sensor 51 and the illuminance sensor. Furthermore, for example, when an application for measuring the sleep time of user U1 is installed in information terminal 7, as the timing when user U1 starts sleeping, determiner 12 may determine the time which the application estimates as the time when user U1 has slept. In any of these modes, determination system 100 can automatically determine the timing at which user U1 starts sleeping.

Outputter 13 outputs information based on the result of determination by determiner 12. In the embodiment, outputter 13 includes control information generator 131, and outputs (transmits) control information (second control signal) generated by the function to air-conditioning apparatus 4 as information based on the result of determination by determiner 12. In other words, based on the result of determination by the determiner, outputter 13 outputs (transmits) the control information (second control signal) to air-conditioning apparatus 4.

Based on the result of determination by determiner 12, control information generator 131 generates the control information (second control signal) of air-conditioning apparatus 4. Here, from an experiment, the present inventor has found that a correlation is found between the Clo value and the temperature (hereinafter, also referred to as “comfort temperature”) of space (room) 2 at which user U1 feels comfortable when user U1 gets up. In the experiment, subjects sleep in spaces (rooms) 2 each having a predetermined environment. The parameters for determining the environment include the set temperature of air-conditioning apparatus 4 and the Clo value. The subjects subjectively declare whether they felt comfortable when they got up. The experiment was performed on a plurality of subjects while the parameters were varied.

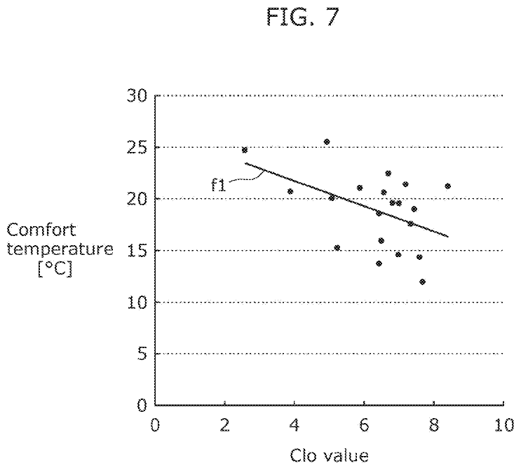

FIG. 7 shows the result of the experiment above. FIG. 7 is a graph showing one example of the correlation between the Clo value and the comfort temperature. In FIG. 7, the ordinate represents the comfort temperature (unit: temperature in Celsius), and the abscissa represents the Clo value. The plotted dots in FIG. 7 are the data of the subjects who declared that they felt “comfortable”. As shown in FIG. 7, the Clo value and the comfort temperature are negatively correlated. In FIG. 7, function f1 is a function obtained through approximation of a large number of pieces of data obtained from the experiment to a linear function. Function f1 may be a quadratic or higher function.

In the embodiment, using function f1 shown in FIG. 7, control information generator 131 calculates the comfort temperature corresponding to the Clo value, which is calculated by determiner 12, and generates the control information (second control signal) including a command to change the set temperature to the calculated comfort temperature. FIG. 3 shows one example of a combination of the Clo value with the corresponding comfort temperature. The comfort temperature shown in FIG. 3 is a natural number and may contain a decimal number. When the calculated comfort temperature is identical to the current set temperature of air-conditioning apparatus 4, control information generator 131 does not generate the control information.

Here, FIG. 7 shows the result of the experiment performed in winter. The sensitivity of a person to the temperature may be varied depending on the season (the outside temperature in particular). Thus, function f1 may be corrected according to the outside temperature. For example, when determination system 100 is used in summer, control information generator 131 may use a function corrected to upwardly shift function f1 shown in FIG. 7 by only a predetermined temperature.

The season can be determined, for example, based on the day and time information obtained from a time server. For example, when the obtained day and time information indicates May to March, the season can be determined as “spring”. When the obtained day and time information indicates June to August, the season can be determined as “summer”. When the obtained day and time information indicates September to November, the season can be determined as “fall”. When the obtained day and time information indicates December to February, the season can be determined as “winter”.

Outputter 13 then outputs (transmits) the control information (second control signal) generated by control information generator 131 to air-conditioning apparatus 4. The control information may be output at a time when control information generator 131 generates the control information or on the day following a day when control information generator 131 generates the control information.

For example, when air-conditioning apparatus 4 operates in the heating mode in turn of seasons from winter to spring, adjustment of the set temperature according to the comfort temperature is also limited. Also when air-conditioning apparatus 4 operates in the cooling mode in turn of seasons from fall to winter, adjustment of the set temperature according to the comfort temperature is also limited. In these cases, i.e., when the calculated comfort temperature reaches the limit temperature, control information generator 131 may generate the control information (second control signal) including a command to switch the operation of air-conditioning apparatus 4. For example, when air-conditioning apparatus 4 currently operates in the heating mode, the control information thus includes a command to switch the operation to the cooling mode. In other words, the control information may include information for switching the operation of air-conditioning apparatus 4 from one of the cooling mode and the heating mode to the other.

When the operation mode of air-conditioning apparatus 4 is switched without permission of user U1, user U1 may feel unnaturalness. For this reason, when the control information is generated, a question signal to inquire whether the operation mode of air-conditioning apparatus 4 is switched may be transmitted to information terminal 7 of user U1. Alternatively, before generation of the control information, an input from user U1 which indicates whether the operation mode of air-conditioning apparatus 4 is switched may be preliminarily accepted through information terminal 7.

Storage 14 is a memory device which stores information (such as a computer program) needed to perform a variety of controls by determiner 12 and outputter 13. Storage 14 is implemented by a semiconductor memory, for example. Any known electronic information storage device can be used without limitation.

### [2. Operation]

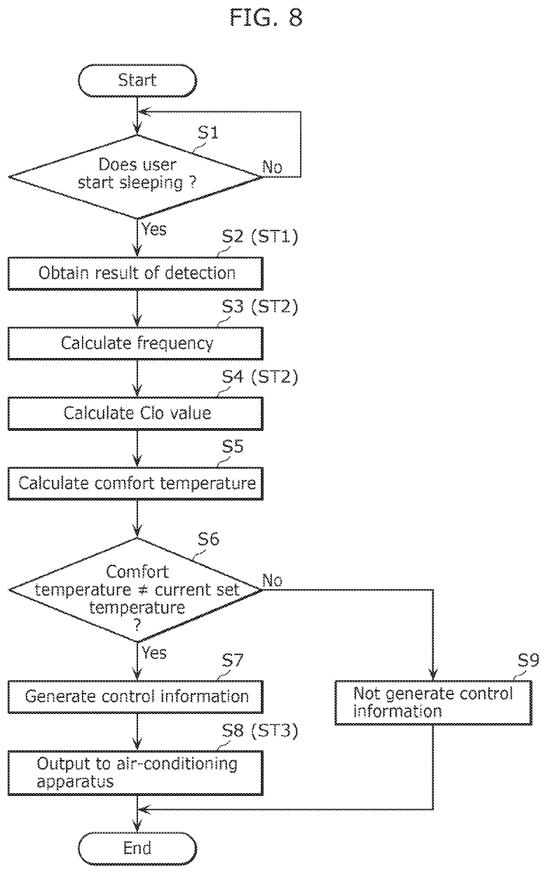

The operation of determination system 100 having such a configuration will be described with reference to FIG. 8 below. FIG. 8 is a flowchart illustrating one example of the operation of determination system 100 according to the embodiment. In the following description, determiner 12 grasps the timing at which user U1 starts sleeping by communicating with information terminal 7.

First, determiner 12 stands by until user U1 starts sleeping (S1: No). When determiner 12 grasps the timing when user U1 starts sleeping (S1: Yes), obtainer 11 obtains the result of detection by first sensor 51 and the result of detection by second sensor 52 by communicating with first sensor 51 and second sensor 52 (S2). Processing S2 corresponds to obtaining step ST1 in the determination method. Processing S2 may be always performed irrespective of the timing when user U1 starts sleeping.

Next, determiner 12 calculates the frequency using the result of detection by first sensor 51 and the result of detection by second sensor 52, which are obtained by obtainer 11 for a predetermined time from the start of sleep of user U1 (S3). Determiner 12 calculates the Clo value corresponding to the calculated frequency with reference to the data shown in FIG. 3 (S4), thereby determining the sleep state of user U1. Processings S3 and S4 correspond to determination step ST2 in the determination method.

Subsequently, using function f1, control information generator 131 calculates the comfort temperature corresponding to the Clo value calculated by determiner 12 (S5). Control information generator 131 then compares the calculated comfort temperature with the current set temperature of air-conditioning apparatus 4 (S6). When the calculated comfort temperature is different from the current set temperature of air-conditioning apparatus 4 (S6: Yes), control information generator 131 generates the control information (second control signal) (S7). Outputter 13 then outputs (transmits) the control information generated by control information generator 131 to air-conditioning apparatus 4 (S8). Processing S8 corresponds to output step ST3 in the determination method. In contrast, when the calculated comfort temperature is identical to the current set temperature of air-conditioning apparatus 4 (S6:No), control information generator 131 does not generate the control information (S9). In this case, outputter 13 does not perform processing S8. Thereafter, this series of processings is repeated.

### 3. Effects

Hereinafter, advantages of determination system 100 according to the embodiment will be described.

As already described, because it is difficult to know the optimal setting of air-conditioning apparatus 4 depending on the cloth worn by user U1 and/or bedding 31 used by user U1, only the basic settings are performed. This results in difficulties of air-conditioning apparatus 4 in providing an environment suitable for user U1.

In contrast, in determination system 100 according to the embodiment, determiner 12 can determine the sleep state of user U1 including covering 6 (the cloth and/or the bedding). For this reason, use of determination system 100 according to the embodiment is advantageous in that provision of an environment suitable for user U1 during sleeping is facilitated in consideration of covering 6 such as the cloth worn by user U1 and/or the bedding used by user U1. For example, by outputting (transmitting) the control information (second control signal) of air-conditioning apparatus 4 based on the result of determination by determiner 12 to air-conditioning apparatus 4, air-conditioning apparatus 4 is controlled to the set temperature in consideration of covering 6. As a result, it is easier to provide an environment ensuring comfortable sleep of user U1.

Another mode rather than determination system 100 according to the embodiment might be considered, in which the input of the information about covering 6 used by user U1 is preliminarily accepted, and air-conditioning apparatus 4 is controlled based on the accepted information. However, this mode is burdensome to user U1 because user U1 should input the information every time when user U1 changes covering 6 to use, for example, in turn of seasons.

Still another mode rather than determination system 100 according to the embodiment might be considered, in which a thermal Image of user U1 and covering 6 during sleeping is obtained with a thermal image sensor, and the sleep state of user U1 is estimated based on the obtained thermal image. However, in this mode, a thermal image sensor, which is a relatively expensive sensor, increases cost.

As disclosed in PTL 1, a mode might be considered, in which using a plurality of relatively inexpensive pyroelectric infrared sensors, the sleep state of user U1 (here, exposure of user U1 from the bedding) is determined from the difference between the body movement amount of the head of user U1 and that of the body thereof. However, in this mode, the arrangement position of the sensor is limited because pyroelectric infrared sensors need to be arranged around the head and the body of user U1. This mode is also burdensome to user U1 because the sensors should be rearranged after bedding 31 is washed or bedding 31 is changed, for example.

In contrast, without sensors separately prepared, determination system 100 according to the embodiment with a simple configuration can determine the sleep state of user U1, for example, using a known infrared sensor included in air-conditioning apparatus 4 as first sensor 51 and an acceleration sensor included in information terminal 7 possessed by user U1 as second sensor 52.

### [4. Modifications]

As above, the embodiment has been described as an example of the technique disclosed in this application. However, the technique in the present disclosure is not limited to this, and can also be applied to embodiments obtained through appropriate modification, replacement, addition, and omission thereof. The components described in the above embodiment can also be combined into a new embodiment.

Thus, modifications of the embodiment will now be illustrated.

### [4-1. Modification 1]

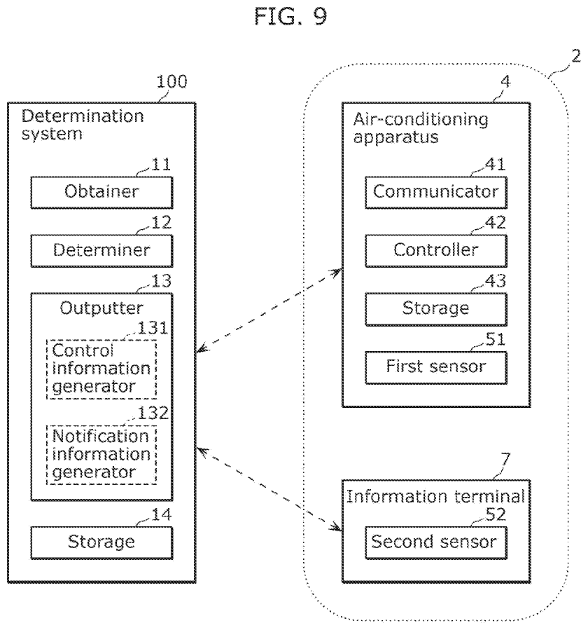

FIG. 9 is a block diagram illustrating the overall configuration of determination system 100 according to Modification 1 of the embodiment. As illustrated in FIG. 9, unlike determination system 100 according to the embodiment, determination system 100 according to the present modification includes outputter 13 including notification information generator 132, and outputs notification information. Specifically, outputter 13 transmits the notification information generated by notification information generator 132 to information terminal 7 of user U1 as information based on the result of determination by determiner 12. In other words, outputter 13 outputs (transmits) the notification information related to the result of determination by determiner 12 to information terminal 7 used by user U1.

The notification information may be a character string and/or picture data displayed on the display of information terminal 7, may be voice data reproduced through the loudspeaker of Information terminal 7, or may be a combination thereof. As one example, the notification information may include information about comparison between the comfort temperature calculated based on the result of determination by determiner 12 and the current set temperature of air-conditioning apparatus 4. In this case, user U1 can adjust the set temperature of air-conditioning apparatus 4 by checking the notification information through information terminal 7.

For example, when the control of air-conditioning apparatus 4 (here, the set temperature) is changed through output (transmission) of the control information (second control signal) to air-conditioning apparatus 4 by outputter 13, outputter 13 may output information indicating that the control of air-conditioning apparatus 4 is to be changed or has been changed as the notification information. In other words, the notification information may include the information related to a change in control of air-conditioning apparatus 4. In this case, user U1 can grasp a change in control of air-conditioning apparatus 4.

For example, in turn of seasons, outputter 13 may output (transmit) information for prompting a change of the cloth worn by user U1 and/or bedding 31 used by user U1 as the notification information. In other words, the notification information may include information for prompting user U1 to change covering 6. For example, in turn of winter to spring, the notification information may include a message saying that “It's time for changing the cloth and the bedding. For better sleep, use a lighter comforter or a smaller number of comforters”. For example, in turn of fall to spring, the notification information may include a message saying that “It's time for changing the cloth and the bedding. For better sleep, use a heavier comforter or more comforters”.

In the present modification, outputter 13 may include only notification information generator 132 without control information generator 131. In other words, in the present modification, outputter 13 may output only the notification information without outputting the control information.

### [4-2. Modification 2]

Unlike determination system 100 according to the embodiment, determination system 100 according to Modification 2 of the embodiment includes determiner 12 which further determines movement of covering 6 as the sleep state. Here, the movement of covering 6 may include movement of the second bedding member such as a cotton blanket or a comforter from a position of the second bedding member in which user U1 is covered therewith or movement thereof to a position of the second bedding member in which user U1 is covered therewith, for example.

Specifically, determiner 12 calculates the frequency at every predetermined time (interval) in a period from sleep of user U1 to wake-up thereof. When the period from sleep of user U1 to wake-up thereof includes both a zone where the frequency is higher than or equal to a predetermined value (e.g., 50%) and a zone where the frequency is less than the predetermined value, determiner 12 determines that covering 6 (second bedding member) is moved.

Here, when determiner 12 determines that covering 6 is moved, it is considered that user U1 unconsciously feels that the environment of space (room) 2 is obstructive to good sleep. In such a case, outputter 13 may change the control of air-conditioning apparatus 4 for good sleep of user U1, for example, by generating the control information (second control signal) by control information generator 131 and outputting (transmitting) the control information to air-conditioning apparatus 4. In such a case, outputter 13 may prompt user U1 to change covering 6 for good sleep of user U1, for example, by generating the notification information by notification information generator 132 and outputting the notification information to information terminal 7.

### [4-3. Other Modifications]

Although in the above embodiment, outputter 13 outputs (transmits) the control information (second control signal), which includes a command to change the set temperature of air-conditioning apparatus 4, to air-conditioning apparatus 4, any other configuration can be used. For example, outputter 13 may output control information to air-conditioning apparatus 4, the control information including to a command to change the wind amount or wind direction of air-conditioning apparatus 4, rather than the command to change the set temperature of air-conditioning apparatus 4. Alternatively, outputter 13 may output control information to air-conditioning apparatus 4, the control Information including at least two parameters among the set temperature, the wind amount, and the wind direction of air-conditioning apparatus 4.

Although in the above embodiment, determination system 100 determines the sleep state of user U1 in room 2 where air-conditioning apparatus 4 is disposed, and controls air-conditioning apparatus 4 based on the result of determination, any other configuration can be used. For example, determination system 100 may be used to determine the sleep state of user U1 in room 2 without air-conditioning apparatus 4. In this case, determination system 100 may prompt a change of the cloth worn by user U1 and/or bedding 31 used by user U1 based on the result of determination as in Modification 1, rather than control air-conditioning apparatus 4 based on the result of determination.

Although in the above embodiment, first sensor 51 is a sensor included in air-conditioning apparatus 4, any other configuration can be used. For example, when air-conditioning apparatus 4 is configured not to include first sensor 51, first sensor 51 may be disposed in room 2 separated from air-conditioning apparatus 4.

Although in the above embodiment, second sensor 52 is a sensor included in information terminal 7 possessed by user U1, any other configuration can be used. For example, when information terminal 7 is configured not to include second sensor 52 or user U1 sleeps while information terminal 7 is not placed therearound, second sensor 52 may be disposed in room 2 separated from information terminal 7.

Although in the above embodiment, second sensor 52 is an acceleration sensor, any other configuration can be used. For example, second sensor 52 may be a piezoelectric sensor. In this case, second sensor 52 may be disposed on the first bedding member such as a mattress.

Although in the above embodiment, determination system 100 determines the sleep state of one user U1, any other configuration can be used. For example, determination system 100 may determine the sleep states of several users U1. In this case, because a corresponding number of rooms 2 to users U1 is present, determination system 100 may obtain the result of detection from first sensor 51 and second sensor 52 for each of rooms 2.

Although in the above embodiment, one first sensor 51 is used, two or more first sensors may be used. Although in the above embodiment, one second sensor 52 is used, two or more second sensors may be used.

Although in the above embodiment, determination system 100 is implemented as a single apparatus, for example, determination system 100 may be implemented with a plurality of apparatuses. When determination system 100 is implemented with a plurality of apparatuses, the components included in determination system 100 may be distributed to the plurality of apparatuses in any manner. In other words, the present disclosure may be implemented by cloud computing, or may be implemented by edge computing.

For example, in the above embodiment, all or part of the components in determination system 100 according to the present disclosure may be configured with dedicated hardware, or may be implemented by executing software programs suitable for the respective components. The components may be implemented by a program executor, such as a central processing unit (CPU) or a processor, which reads out and executes a software program recorded in a hard disk drive (HDD) or a recording medium such as a semiconductor memory.

The components in determination system 100 according to the present disclosure may be configured with one electronic circuit or a plurality of electronic circuits. The one electronic circuit or the plurality of electronic circuits may each be a general-purpose circuit, or may be a dedicated circuit.

The one electronic circuit or the plurality of electronic circuits may include a semiconductor device, an integrated circuit (IC), or large scale integration (LSI), for example. The IC or LSI may be integrated into a single chip, or may be integrated into a plurality of chips. Here, although these electronic circuits are referred to as IC or LSI, they may be referred to as system LSI, very large scale integration (VLSI), or ultra large scale integration (ULSI) depending on the degree of integration. Moreover, a field programmable gate array (FPGA) programmable after fabrication of LSI can also be used for the same purpose.

Moreover, general or specific aspects of the present disclosure may be implemented with a system, an apparatus, a method, an integrated circuit, or a computer program. Alternatively, general or specific aspects of the present disclosure may be implemented with a non-transitory computer-readable recording medium having the computer program recorded thereon, such as an optical disc, a HDD, or a semiconductor memory. For example, the present disclosure may be implemented as a program for causing a computer to execute the control method according to the above embodiment. Moreover, this program may be recorded on a non-transitory recording medium such as a computer-readable CD-ROM, or may be distributed via a communication path such as the Internet.

As above, the embodiment has been described as an illustration of the technique according to the present disclosure. For this purpose, the accompanied drawings and detailed description have been provided.

Accordingly, the components described in the accompanied drawings and the detailed description may include not only components essential for solving the problems but also those not essential for solving the problems, which components are described for illustration of the above technique. For this reason, it should not be immediately approved that those non-essential components are essential because those non-essential components are described in the accompanied drawings and the detailed description.

Moreover, the above embodiment is an illustration of the technique according to the present disclosure, and thus, can be subjected to a variety of modifications, replacements, additions, and omissions in the scope of claims or equivalents thereof.

### SUMMARY

As described above, determination system 100 according to the embodiment includes obtainer 11, determiner 12, and outputter 13. Obtainer 11 obtains a result of detection by first sensor 51 which detects a body movement of a body part of user U1 exposed from covering 6, and a result of detection by second sensor 52 which detects a body movement of user U1. Determiner 12 determines a sleep state of user U1 including covering 6, based on the result of detection by first sensor 51 and the result of detection by second sensor 52, which are obtained by obtainer 11. Outputter 13 outputs information based on a result of determination of determiner 12.

Such a configuration is advantageous in that provision of an environment suitable for user U1 during sleeping is facilitated in consideration of covering 6 such as the cloth worn by user U1 and/or the bedding used by user U1.

Moreover, for example, determiner 12 determines the sleep state of user U1, based on a result of comparison between a frequency of detection of the body movement of user U1 by second sensor 52 and a frequency of detection of the body movement of user U1 by first sensor 51.

Such a configuration is advantageous in that the state of covering 6 during sleeping is easy to determine.

Moreover, for example, outputter 13 outputs control information to air-conditioning apparatus 4, based on a result of determination by determiner 12.

Such a configuration is advantageous in that provision of an environment suitable for user U1 during sleeping is facilitated by controlling the temperature of space 2 to a temperature at which user U1 is more likely to feel comfortable during sleeping.

Moreover, for example, the control information includes information for switching the operation of air-conditioning apparatus 4 from one of the cooling mode and the heating mode to the other.

Such a configuration is advantageous in that even when the control of the temperature of space 2 in one of the cooling mod and the heating mode reaches a limit, by switching to the other operation mode, the temperature of space 2 is easy to control to a temperature at which user U1 is more likely to feel comfortable during sleeping.

Moreover, for example, outputter 13 outputs notification information related to the result of determination by determiner 12 to information terminal 7 used by user U1.

Such a configuration is advantageous in that the sleep state is more easily grasped by checking the notification information using information terminal 7 by user U1.

Moreover, for example, the notification information includes information related to control of air-conditioning apparatus 4.

Such a configuration is advantageous in that it is less likely that user U1 feels uncomfortable by informing user U1 about a change in control of air-conditioning apparatus 4.

Moreover, for example, the notification information includes information for prompting user U1 to change covering 6.

Such a configuration is advantageous in that user U1 is more likely to use covering 6 suitable for user U1, facilitating provision of an environment suitable for user U1 during sleeping.

Moreover, for example, determiner 12 further determines, as the sleep state, whether covering 6 is moved.

Such a configuration is advantageous in that not only the type of covering 6 but also the degree of satisfaction with covering 6 which user U1 has can be indirectly determined, facilitating more detailed determination of the sleep state of user U1.

Moreover, for example, first sensor 51 is a pyroelectric infrared sensor, and second sensor 52 is a vibration sensor, an acceleration sensor, or a piezoelectric sensor.

Such a configuration is advantageous in that compared to the case where a thermal image sensor is used, for example, the sleep state of user U1 Is determined with a simple and inexpensive configuration.

Moreover, for example, determination system 100 according to the embodiment may further include second sensor 52.

Such a configuration is advantageous in that the user does not need to prepare second sensor 52.

Moreover, the sensor (second sensor 52) according to the embodiment has a communication function to communicate with determination system 100 and a detection function to detect a body movement of user U1. The sensor transmits a result of detection using the detection function to determination system 100 using the communication function.

Moreover, the determination method according to the embodiment includes obtaining step ST1, determination step ST2, and output step ST3. Obtaining step ST1 includes obtaining a result of detection by first sensor 51 which detects a body movement of a body part of user U1 exposed from covering 6, and a result of detection by second sensor 52 which detects a body movement of user U1. Determination step ST2 includes determining a sleep state of user U1 including covering 6, based on the result of detection by first sensor 51 and the result of detection by second sensor 52, which are obtained in obtaining step ST1. Output step ST3 includes outputting information based on a result of determination in determination step ST2.

Such a configuration is advantageous in that provision of an environment suitable for user U1 during sleeping is facilitated in consideration of covering 6 such as the cloth worn by user U1 and/or the bedding used by user U1.

Moreover, a program according to the embodiment causes one or more processors to execute the above determination method.

Such a configuration is advantageous in that provision of an environment suitable for user U1 during sleeping is facilitated in consideration of covering 6 such as the cloth worn by user U1 and/or the bedding used by user U1.

### INDUSTRIAL APPLICABILITY

The present disclosure can be used in determination systems which determine the state of users during sleeping.

1. A determination system comprising:
an obtainer which obtains a result of detection by a first sensor which detects a body movement of a body part of a user exposed from a covering, and a result of detection by a second sensor which detects a body movement of the user;
a determiner which determines a sleep state of the user, based on the result of detection by the first sensor and the result of detection by the second sensor, which are obtained by the obtainer, the sleep state including a state of the covering; and
an outputter which outputs information based on a result of determination by the determiner.

2. The determination system according to claim 1,
wherein the determiner determines the sleep state of the user, based on a result of comparison between a frequency of detection of the body movement of the user by the second sensor and a frequency of detection of the body movement of the user by the first sensor.

3. The determination system according to claim 1,
wherein the outputter outputs control information to an air-conditioning apparatus, based on the result of determination by the determiner.

4. The determination system according to claim 3,
wherein the control information includes information for switching an operation of the air-conditioning apparatus from one of a cooling mode and a heating mode to a remaining one of the cooling mode and the heating mode.

5. The determination system according to claim 1,
wherein the outputter outputs notification information related to the result of determination by the determiner to an information terminal used by the user.

6. The determination system according to claim 5,
wherein the notification information includes information related to a change in control of the air-conditioning apparatus.

7. The determination system according to claim 5,
wherein the notification information includes information for prompting the user to change the covering.

8. The determination system according to claim 1,
wherein the determiner further determines, as the sleep state, whether the covering is moved.

9. The determination system according to claim 1,
wherein the first sensor is a pyroelectric infrared sensor, and
the second sensor is a vibration sensor, an acceleration sensor, or a piezoelectric sensor.

10. The determination system according to claim 1, further comprising the second sensor.

11. A sensor having a communication function to communicate with the determination system according to claim 1, and a detection function to detect the body movement of the user,
wherein the sensor transmits a result of detection using the detection function to the determination system using the communication function.

12. A determination method comprising:
obtaining a result of detection by a first sensor which detects a body movement of a body part of a user exposed from a covering, and a result of detection by a second sensor which detects a body movement of the user;
determining a sleep state of the user, based on the result of detection by the first sensor and the result of detection by the second sensor, which are obtained in the obtaining, the sleep state including a state of the covering; and
outputting a result of determination in the determining.

13. A non-transitory computer-readable recording medium for use in a computer, the recording medium having a computer program recorded thereon for causing one or more processors to execute the determination method according to claim 12.
